(19) United States
(12) Patent Application Publication
(10) Pub. No.: US 2003/0124180 A1
(43) Pub. Date: Jul. 3, 2003

(54) Liposomes containing active substances

(76) Inventors: Jurgen Ebert, Berlin (DE); Gerd Berger, Berlin (DE)

Correspondence Address: Friedrich Kueffner, Suite 910, 317 Madison Avenue, New York, NY, 10017, US

(21) Appl. No.: 10/258,997

(22) PCT Filed: May 2, 2001
(86) PCT No.: PCT/EP01/04900

(30) Foreign Application Priority Data
May 2, 2000 (DE) 100 21 030.9

(51) Int. Cl. A61K 9/127; A61K 49/00

(52) U.S. Cl. 424/450; 424/9.321; 424/9.51

(57) ABSTRACT

Active ingredients encapsulated in liposomes which are characterized in that the active ingredient(s) are encapsulated in a proportion of from 1% by weight to 90% by weight based on the amount of active ingredient employed are claimed. These active ingredients are suitable in particular for the treatment of tumors, as food supplements, for producing contrast agents for imaging methods and for producing diagnostic agents for diseases.

The present invention relates to liposomes which comprise active ingredients, in particular active pharmaceutical ingredients, to a process for their preparation, and to the use of these liposomes for the manufacture of pharmaceutical products.

The administration of active pharmaceutical ingredients, in particular of large active ingredient molecules, is frequently problematic. The active ingredient is distributed in different amounts, depending on its solubility in water or in lipophilic solvents, in individual regions of the body, and frequently only small amounts of active ingredient reach the target organ. It is moreover possible for the active ingredients to be metabolized through reactions with substances present in the body, and unwanted side effects for the whole body may result.

Liposomes are employed as carrier substances in particular for relatively large active ingredient molecules. The active ingredients are enclosed in the liposomes and cannot be attacked by other substances. It is moreover possible to construct the liposomes in such a way that the medicaments enclosed therein direct, via a specific structure of the liposome cell, into particular target organs.

Liposomes are artificially prepared spherical lipid vesicles consisting of one or more concentric liquid bilayers with aqueous interior. They can be prepared by finely dividing (dispersing) phospholipids mechanically in aqueous media. Multilamellar vesicles with a diameter of about 0.1 to 5 μm are distinguished from unilamellar vesicles with a size of from 0.02 to 0.05 μm or about 1 μm. Liposomes play an important part as carrier systems in particular in the treatment of tumors. An example of an active ingredient which is administered encapsulated in liposomes is paclitaxel. Paclitaxel has only low solubility in water and is therefore frequently used in combination with solubilizers such as Cremophor (polyethoxylated castor oil). The use of this combination leads, however, to considerable side effects such as, for example, anaphylactoid reactions. Dilution of such solutions with physiological saline for administration has the disadvantage that paclitaxel has inadequate stability in physiological saline.

The international patent application WO 93/18751 discloses paclitaxel liposome combinations from which, in particular, vesicles with a positive charge based on cardiolipin, phosphatidylcholine and cholesterol were prepared. However, it was necessary for the prepared liposomes, as shown by the examples, to be administered in animal experiments on four consecutive days, from which it may be concluded that the desired antitumor effect was not achievable after a single dose.

A problem which frequently arises also when other active ingredients are encapsulated in liposomes is that the liposomes in some cases lead to side effects or they reach the liver directly, where they are promptly eliminated.

Wide-ranging research with the intention of increasing the capacity of liposomes has not led to the desired effects because the liposomes do not themselves reach the target organ.

The present invention was accordingly based on the object of providing a liposomal system in which it is possible to accumulate the activity of active ingredients in the treatment of diseases, or the accumulation of active ingredients and/or pharmaceutical substances which are to be accumulated in a particular target organ, such as, for example, contrast agents for imaging methods etc. It was intended to increase the efficacy of such systems in the treatment of diseases etc. and preferably also to reduce the number of side effects.

It has been found, surprisingly, that the effect of active ingredients encapsulated in liposomes, such as antitumor agents, can be increased many times if the active ingredient is not completely encapsulated in liposomes, i.e. if free active ingredient molecules are still present outside the liposomes, in the solution/emulsion to be administered.

The present invention accordingly relates to an active ingredient encapsulated in liposomes, which is characterized in that it is encapsulated in a proportion of from 1% by weight to 90% by weight based on the amount of active ingredient employed.

For the purposes of the present invention, the proportion encapsulated means that encapsulated active ingredient molecules are present in the center of the liposomes or in the membrane, in contrast to free active ingredient molecules which remain in the solvent surrounding the liposomes or are loosely bound to the surface of the lipid membranes by van-der-Waal's interactions.

Active ingredients which can be employed in the present invention are medicaments and pharmacologically active substances, nutrients, cosmetic substances, diagnostic substances and contrast agents for imaging methods. The term active ingredient also encompasses the pharmacologically acceptable salts of the active ingredients. Suitable compounds are lipophilic and amphiphilic molecules, i.e. substances which are insoluble in water or have only low solubility in water, and which can be prepared in the form of emulsions or micellar preparations. Examples of suitable medicaments are anticancer agents, antitumor agents, antibiotics, antimicotic, antivirual, anthelmintic and antiparasitic compounds.

Compounds which are suitable to be employed for the treatment of tumors or cancerous growth are anthracyclines such as doxorubicin, daunorubicin, carinomycin, N-acetylatriamycin, rubidacon, 5-imido-daunomycin, N-acetyldaunomycin, pirarubicin and epirubicin, alkaloids such as vincristine, vinblastine, etoposide, ellepticin and damptothecin. Further suitable substances are 5-fluorouracil, taxanes such as paclitaxel (Taxol®) and derivatives of paclitaxel, e.g. taxotere (Docetaxol®) or mitrotan, platinum compounds such as cisplatin, carboplatin, lobaplatin and phenestrin).

Examples of anti-inflammatory agents which can be employed in the present invention are steroidal and non-steroidal and other anti-inflammatory compounds such as prednisone, methyl-prednisolone, paramethazone, 11-fluorocortisol, triamciniolone, betamethasone and dexamethasone, ibuprophen, piroxicam, beclomethasone, methotrexate, acaridine, etritinate, anthraline, psoralines, salycilates such aspirin, and immuno-suppressants such as cyclosporin. In particular, anti-inflammatory corticosteorids, and cyclosporin which has an anti-inflammatory and immunosuppressant activity, are lipophilic compounds which can be employed particularly advantageously in the present invention.

Further pharmacologically active substances which can be employed in the present invention are anesthetics such as methoxyflurane, isoflurane, N-flurane, halothane and bezocaine, antiulcer agents such as cimetidine, spasmolytic agents such as barbiturates, azothioprine, an immunosuppressant and antirheumatic agent, and muscle relaxants such as dantrolene and diazepam.

Processes for preparing lipophilic derivatives which can be prepared in the form of liposomes or mycelles are well known to the skilled worker.

Examples of components which can be employed as contrast agents in imaging methods are contrast agents for ultrasonic, X-ray and NMR methods. Particularly suitable for X-ray methods are radioisotopes or compounds which comprise such radioisotopes, such as, for example, iodine, octanes, halohydrocarbon and Renografin. Suitable contrast agents (gadolinium) which can be employed in NMR methods may be lipid-soluble paramagnetic compounds.

Food supplements which can be incorporated into the liposomal system of the present invention are amino acids, sucrose, proteins, carbohydrates, lipid-soluble vitamins, fats (lipids). Combinations of various food supplements are likewise suitable.

Further suitable substances are immunomodulators and vaccines.

The active ingredients employed can be encapsulated in a manner known per se with addition of liposomes. In order to adjust the desired degree of encapsulation it is possible to encapsulate only part of the active ingredient. The partial encapsulation can take place through use of appropriate starting materials or suitable process conditions. It is possible in a further embodiment for active ingredient which is completely or virtually completely encapsulated in liposomes to be mixed with the appropriate amount of unencapsulated active ingredient or solutions/emulsions thereof, so that the desired degree of encapsulation is obtained. Overall, the degree of encapsulation can be adjusted by simple alterations to the process.

The liposome has a closed structure which consists of a lipid bilayer which encloses an aqueous inner core. The liposomes employed according to the invention are not restricted to particular liposomes; it is possible to employ both neutral, negatively or positively charged liposomes which may have a unilamellar, i.e. composed of one lipid bilayer, or polylamellar, i.e. composed of a plurality of lipid bilayers, structure and be prepared by methods known to the skilled worker.

The liposomes are normally prepared from phospholipids and, where appropriate, cholesterol and cholesterol derivatives and/or one or more hydrophilic, lipophilic or amphiphilic component(s).

Examples of suitable phospholipids are phosphatidylcholine (PC), distearoylphsphatidylcholine (DSPC), soybean phosphatidylcholine (soya PC), hydrogenated soybean phosphatidylcholine (HSPC), egg phosphatidylcholine (egg PC), hydrogenated egg phosphatidylcholine (HEPC), dipalmitoylphosphatidylcholine (DPPC), dimyristoylphosphatidylcholine (DMPC) and any mixtures thereof, it being possible for these substances to be synthetic, semisynthetic or natural products.

Further suitable phospholipids are phosphatidyl glycerides (PG) and phosphatidic acid, such as dimyristoylphosphatidyl glyceride (DMPG), dilaurylphosphatidyl glyceride (DLPG), dipalmitoylphosphatidyl glyceride (DPPG), distearoylphosphatidyl glyceride (DSPG), dimyristoylphosphatidic acid (DMPA), dilaurylphosphatidic acid (DLPA), dipalmitoylphosphatidic acid (DPPA), distearoylphosphatidic acid (DSPA), and phosphatidylethanolamines, phosphatidylinositoles and phosphatidic acid, which comprise residues of lauric acid, myristic acid, stearic acid and/or palmitic acid.

Preferred phospholipids are HSPC, DSPC and HEPC.

Positively charged liposomes can be formed for example from a solution which contains phosphatidylcholine, cholesterol and stearylamine. Negatively charged liposomes can be obtained for example from solutions which [lacuna] phophatidylcholon, cholesterol and phosphatidylserine or, preferably, cardiolipin. The skilled worker is aware that other additives can also be added in order to modify the properties of the liposomes obtained.

The properties of liposomes can be altered for example by adding α-tocopherol. Good results are also obtained with so-called PEG liposomes, i.e. liposomes with polyethylene glycol chains (PEG) in the lipid layer, it being possible for the PEG either to be bound in the molecule of the phospholipids or to be present as free substance. The molecular weight of the PEG chains is preferably between 400 and 20 000, particularly preferably between 600 and 10 000 and, in particular, between 600 and 5 000.

The encapsulating agents mentioned in WO96/05821 can also be employed as liposomes.

Liposomes which are preferably employed are formed, for example, from HSPC, DSPC and/or HEPC as phospholipid, cholesterol and/or N-(O-methyl-poly(ethylene glycol)-1,2-distearoyl-sn-glycero-3-phosphoethanolamine and, where appropriate, polyethylene glycol.

The ratio of lipid to active ingredient can be adjusted within wide ranges and usually depends on the active ingredient used and on the lipid employed to prepare the liposome, and the other components. In one possible embodiment, the lipid and active ingredient are present in the finished encapsulated product in a ratio of amounts of from 5:1 to 100:1, preferably from 10:1 to 40:1, in particular from 15:1 to 25:1. If cholesterol or a cholesterol derivative is also present, the molar ratio of lipid to cholesterol is preferably from 3:1 to 1:3, preferably from 2:1 to 1:2. Polyethylene glycol is preferably employed in an amount of from 1 to 50 mol %, in particular from 5 to 20 mol %, based on the lipid.

The size of the active ingredients encapsulated in liposomes according to the invention is preferably less than 200 nm, particularly preferably less than 150 nm.

The physiological tolerability and efficacy of the active ingredients encapsulated in liposomes according to the invention can be further improved through combination with suitable pharmaceutical carriers. Examples of particularly suitable pharmaceutical carriers are, for example, so-called microspheres which are preferably prepared from polysacharides or polysacharide derivatives and particularly preferably are degradable. The polysacharides are preferably selected from starch and starch derivatives, gelatin, albomin, colagen, dextran, dextan derivatives or similar materials. Lyopohilized or degradable starch or gelatin particles are particularly preferred. Such microspheres preferably have a water-insoluble, but hydrophilic, three-dimensional network, which is swellable in water, of polysacharide molecules or corresponding derivatives. Suitable microspheres are described in DE-US 25 24 279, WO 88/09163 and WO 89/03207. The diameter of the microspheres is preferably between 0.1 and 200 μm, in particular between 10 and 100 μm.

The liposomal systems prepared according to the invention can be put directly into the bloodstream of the people to be treated, and intraperitoneal, subcutaneous or inhalational administration is also possible.

The liposomes employed according to the invention can be prepared by any process known in the prior art. For example, the liposomes can be dissolved in a suitable solvent, normally in a nonpolar or only weakly polar solvent which can be removed again without leaving harmful substances behind, such as ethanol, methanol, chloroform, butanol or acetone. If active ingredient mixtures are employed, the individual solutions can also be mixed together. The lipophilic material is also dissolved and mixed with the active ingredient-containing solution. After removal of the solvent, for example by a lyophilization process, the lipid film remains on the active ingredient. The mixture can be stored in this form, where appropriate under an inert gas atmosphere, with particular preference for low storage temperatures, such as at −20° C. Formation of the liposomes normally takes place by adding a suitable solution to the lipid film. Typical solutions are polar solutions, preferably aqueous salt solutions, such as isotonic saline. The liposomes are formed for example by mixing, such as by mixing in a vortex (vortexing). If small vesicles are prepared, such as unilamellar vesicles, the solution can also be treated with ultrasound.

In a further embodiment of the present invention, it is also possible to employ mixtures of multilamellar vesicles and unilamellar vesicles.

The prepared liposomes can be administered directly to the patient or be stored under suitable conditions. The lipophilic active ingredients are preferably stored as dry substances covered with lipid films at about −20° C. After hydration, liposome suspensions to which a suitable amount of unencapsulated active ingredient has been added can be stored in buffered or neutral saline solutions over a period of from a few hours up to months, depending on the temperature and the ingredients. The liposomal active ingredient system of the present invention can be administered in amounts of from about 5 to 150 mg of active ingredient/kg of body weight of the patient within a period of from 1 minute to 5 hours.

The present invention further relates to the use of an active ingredient which has been encapsulated in liposomes as described above for producing a medicament for the treatment of tumors, as food supplement, for preparing a contrast agent for imaging methods and for preparing a diagnostic agent for diseases.

### EXAMPLES

Preparation of the Liposomes:

Hydrogenated egg phosphatidylcholine (HEPC, 50 mg/ml; Nattermann Phospholipid GmbH, Cologne, Germany), cholesterol (CH, 24.8 g/ml; Merck, Darmstadt, Germany) and polyethylene glycol (MPEG-DSPE, 3000, 5.4 mg/ml; Sygena LTD, Liestal, Switzerland) are dissolved in chloroform in the molar ratio 1:1:0.1. The organic phase is removed in a rotary evaporator, and the resulting well-dried lipid film is resuspended with an active ingredient solution and a Gd-DTPA FS solution. This liposome dispersion is shaken at room temperature for 24 h, and the multilamellar vesicles (MLV) produced thereby are subjected to sonification (hose sonifier, 6×4 min with 50% intensity, Branson B225 sonifer; Branson, Carouge-Geheve, Switzerland) and subsequently centrifuged (3 000 rpm for 20 min) in order to remove abraded titanium from the liposome dispersion.

The active ingredients used were 5-fluorouracil (Ribolour® 1000; 50 mg/ml infusion solution, Ribosepharm GmbH, Munich, Germany) and carboplatin (Ribocarabo® 50 mg/ml infusion solution, Ribosepharm GmbH, Munich, Germany).

The size of the vesicles was determined using a photon correlation spectrometer (Coulter Counter N4 MD modell and the AccuComp® System, Coulter Electronics Inc. Hialeah, US) and for both active ingredients varied over sizes of the order of 100+50 nm.

The 5-fluorouracil liposomes were also investigated by nuclear magnetic resonance spectroscopy, namely H1-NMR and NOESY spectroscopy (NOE=nuclear Overhauser effect). The H1-NMR spectrum of the 5-fluorouracil (c=50 mg/ml) encapsulated in liposomes showed in addition to the H6 proton of 5-FU at 7.5 ppm also signals of the liposomal PEG matrix at between 2.5 and 4.0 ppm. The 1D-NOE spectrum (selective excitation of the H6 proton at 7.5 ppm) revealed a positive NOE resonance at 3.5 ppm, which signifies spatial closeness of the active ingredient to the liposome, i.e. partial encapsulation of the active ingredient.

The active ingredients were in each case administered in free form intravenously and intra-arterially and in encapsulated form with and without starch microspheres (Spherex®, Pharmacia Upjohn) in general for tumor treatment of liver tumors.

In the antitumor system, the tumor targeting was effected by liposomal encapsulation of the cytostatics and addition of starch microspheres. The SUV-PEG liposomes used have a particle size of 113 nm ±25 nm, and the starch microspheres average 45 μm.

The active ingredient concentrations were in the tumor and in the liver were measured in AUC (area under the curve). The results are shown in Table 1.

TABLE 1

| | Liver | Tumor |
|---|---|---|
| Carboplatin (CPT) | 147,980 | 87,950 |
| CPT encapsulated | 50,060 | 434,200 |
| according to the | | |
| invention | | |
| 5-Fluorouracil | 27,822 | 62,655 |
| (5-FU) | | |
| 5-FU encapsulated | 35,842 | 272,886 |
| according to the | | |
| invention | | |

1. An active ingredient encapsulated in liposomes which is characterized in that it is encapsulated in a proportion of from 1% by weight to 90% by weight based on the amount of active ingredient employed.

2. An active ingredient encapsulated in liposomes as claimed in claim 1, characterized in that the active ingredient is encapsulated in a proportion of from 5 to 85% by weight based on the amount of active ingredient employed.

3. An active ingredient encapsulated in liposomes as claimed in either of claims 1 or 2, characterized in that the active ingredient is selected from medicaments and pharmacologically active substances, nutrients, cosmetic substances, diagnostic substances and contrast agents for imaging methods.

4. An active ingredient encapsulated in liposomes as claimed in any of claims 1 to 3, characterized in that the liposomes are formed from phospholipids and, where appropriate, cholesterol and cholesterol derivatives and/or one or more hydrophilic, lipophilic or amphiphilic component(s).

5. An active ingredient encapsulated in liposomes as claimed in claim 4, characterized in that the phospholipid is selected from phosphatidylcholine (PC), distearoylphosphatidylcholine (DSPC), soybean phosphatidylcholine (soya-PC), hydrogenated soybean phosphatidylcholine (HSPC), egg phosphatidylcholine (egg PC), hydrogenated egg phosphatidylcholine (HEPC), dipalmitoylphosphatidylcholine (DPPC), dimyristoylphosphatidylcholine (DMPC) and any mixtures thereof.

6. The use of the active ingredient encapsulated in liposomes as claimed in any of claims 1 to 5 for producing a medicament for the treatment of tumors.

7. The use as claimed in claim 6, characterized in that the active ingredient is employed in combination with a pharmaceutical carrier, in particular with microspheres which are preferably prepared from polysacharides or polysacharide derivatives and are particularly preferably degradable.

8. The use of the active ingredient encapsulated in liposomes as claimed in any of claims 1 to 5 as food supplement.

9. The use of the active ingredient encapsulated in liposomes as claimed in any of claims 1 to 5 for producing a contrast agent for imaging methods.

10. The use of the active ingredient encapsulated in liposomes as claimed in any of claims 1 to 5 for producing a diagnostic agent for diseases.
